(19) United States
(12) Patent Application Publication
(10) Pub. No.: US 2007/0215472 A1
(43) Pub. Date: Sep. 20, 2007

(54) Electroosmotic flow for end labelled free solution electrophoresis

(76) Inventors: Gary W. Slater, Ottawa (CA); Laurette C. McCormick, Ottawa (CA)

Correspondence Address: KIRBY EADES GALE BAKER, BOX 3432, STATION D, OTTAWA, ON, K1P 6N9, CA

(21) Appl. No.: 11/724,294

(22) Filed: Mar. 15, 2007

Related U.S. Application Data
(60) Provisional application No. 60/782,272, filed on Mar. 15, 2006.

(51) Int. Cl. C07K 1/26; G01N 27/00

(52) U.S. Cl. 204/451; 204/601

(57) ABSTRACT

End Labelled Free Solution Electrophoresis (ELFSE) provides a means of separating polymer molecules such as ssDNA according to their size, via free solution electrophoresis, thus eliminating the need for polymer separation via gels or polymer matrices. Here, significant improvements in ELFSE are disclosed via concurrent exposure of the polymer molecules to an electroosmotic flow. When the methods are applied to DNA sequencing by ELFSE, significant improvements in read length are observed.

### CROSS REFERENCE TO RELATED APPLICATION

This application claims the priority right of prior U.S. patent application 60/782,272 filed Mar. 15, 2006 by applicants herein.

### FIELD OF THE INVENTION

The invention relates to the field of polymer separation. More particularly, the invention relates to the separation of polymer molecules of different sizes.

### BACKGROUND TO THE INVENTION

Techniques for separation of polymer molecules on the basis of their size are well known in the art. For example, polynucleotides or polypeptides may be separated via gel-based electrophoresis techniques, which involve gel matrices comprising for example agarose or polyacrylamide. In the case of DNA sequencing, polynucleotides may be separated with a resolution as low as a single polymer unit (nucleotide).

In one example, End Labelled Free Solution Electrophoresis (ELFSE) provides a means of separating polymer molecules such as DNA with free solution electrophoresis, eliminating the need for gels and polymer solutions. In free solution electrophoresis, DNA is normally free-draining and all fragments elute at the same time. In contrast, ELFSE often uses uncharged label molecules attached to each DNA fragment in order to render the electrophoretic mobility of the DNA fragments size-dependent. For example, methods for ELFSE are disclosed for example in U.S. Pat. Nos. 5,470,705, 5,514,543, 5,580,732, 5,624,800, 5,703,222, 5,777,096, 5,807,682, and 5,989,871, all of which are incorporated herein by reference. Many types and variations of end labels are known in the art, as described in the aforementioned patents, as well as United States patent publication US2006/0177840 published May 1, 2006, which is also incorporated herein by reference.

With ELFSE, however, the larger molecules can move too quickly resulting in insufficient separation, thereby limiting the read-length of the DNA. In contrast, smaller molecules can sometimes be over-separated, increasing the time required for the sequencing.

It follows that there remains a need to develop further improved methods for polymer separation. For example, there remains a need to develop methods for DNA sequencing that avoid any requirement for gels or polymer solutions, and avoid the disadvantages presented by traditional ELSFE techniques that are known in the art.

### SUMMARY OF THE INVENTION

It is an object of the invention, at least in preferred embodiments, to provide a method for separating polymer molecules on the basis of their size.

It is another object of the invention, at least in preferred embodiments, to provide a method for sequencing DNA.

In one aspect the invention provides a method for separation of polymer molecules in solution according to their relative size, each polymer molecule comprising an end-label at or near one or both ends thereof, the method comprising the steps of:

(1) subjecting the polymer molecules in solution to electrophoresis;

(2) subjecting the polymer molecules in solution during electrophoresis to an electroosmostic flow, such that the polymer molecules migrate in the solution at different rates, and optionally in different directions, according to their mobility in the solution.

Preferably, in step (2) the speed of electroosmotic flow is about equal to a speed of unlabelled DNA subjected to the electrophoresis of step (1). In an alterative aspect, in step (2) the speed of electroosmotic flow is preferably less than a speed of unlabelled DNA subjected to the electrophoresis of step (1).

Preferably, at least some of the polymer molecules migrate in opposite directions according to a relative force upon them caused by said electrophoresis and said electroosmostic flow.

Preferably, said solution is retained in a capillary tube. More preferably, the capillary tube comprises an internal wall that is uniformly charged, and wherein the solution at both ends of the capillary tube is at about the same pressure.

Preferably, in step (2) the electroosmotic flow is constant and causes a countercurrent to a mobility of at least some of the polymer molecules during electrophoresis.

Preferably, the polymer molecules are separated with a polymer unit resolution Sₘ calculated according to equation (8):

$${S}_{m} ( {M}_{c} , {\tilde{μ}}_{EOF} ) ≡ \frac{{FWHM}_{t}}{\vert \frac{\partial t}{\partial {M}_{c}} \vert} \qquad ( 8 )$$

wherein the components of equation 8 are herein defined.

Preferably, the polymer molecules are polynucleotides. More preferably, the polynucleotides are separated with a resolution of one nucleotide or less. More preferably, the polynucleotides are derived from sequencing reactions for a DNA, the method further comprising a step of:

(3) deducing a nucleotide in said DNA corresponding to each polymer molecule, so as to deduce a sequence of the DNA.

In another aspect, the present invention provides for an apparatus for separation of polymer molecules in solution according to their relative size, each polymer molecule comprising an end-label at one or both ends thereof, the apparatus comprising:

(1) electrophoresis means for subjecting the polymer molecules in the solution to electrophoresis;

(2) electroosmostic flow means for subjecting the polymer molecules in the solution during electrophoresis to an electroosmostic flow;

whereupon subjecting the polymer molecules to simultaneous electrophoresis and electroosmotic flow, the polymer molecules migrating in the solution at different rates, and optionally in different directions, according to their mobility in the solution.

In another aspect the invention provides for a method for sequencing a section of a DNA molecule, the method comprising the steps of:

(a) synthesizing a first plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific adenine base in said section of DNA;

(b) synthesizing a second plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific cytosine base in said section of DNA;

(c) synthesizing a third plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific guanine base in said section of DNA;

(d) synthesizing a fourth plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific thymine base in said section of DNA;

(e) attaching a chemical moiety to at least one end nucleotide at or near at least one end of said ssDNA molecules to generate end-labeled ssDNAs; and

(f) subjecting each plurality of ssDNA molecules to free-solution electrophoresis;

(g) subjecting the polymer molecules in solution during electrophoresis to an electroosmostic flow such that the polymer molecules migrate in the solution at different rates, and optionally in different directions, according to their mobility in the solution;

(h) identifying the nucleotide sequence of the section of DNA in accordance with the relative electrophoretic mobilities of the end labeled ssDNAs in each plurality of ssDNAs;

wherein any of steps (a), (b), (c), and (d) may be performed in any order or simultaneously;

whereby each end label imparts increased hydrodynamic friction to at least one end of each end-labeled ssDNA thereby to facilitate separation of the end-labeled ssDNAs according to their electrophoretic mobility.

Preferably, the end labels are uncharged chemical moieties. Preferably, the end labels are selected from among polypeptides and polypeptoids. More preferably, the end labels are selected from the group consisting of Streptavidin, or a derivative thereof, N-methoxyethylglycine (NMEG)-based polymers comprising up to 300 preferably 100 monomer units, and a molecule consisting of a poly(NMEG) backbone optionally grafted with oligo(NMEG) branches. Preferably, the section of DNA comprises less than 2000 nucleotides. More preferably, the section of DNA comprises less than 500 nucleotides. Most preferably, the section of DNA comprises less than 100 nucleotides.

In another aspect the invention provides an apparatus for sequencing a DNA molecule by carrying out at least steps (f), (g), and (h) of the method of claim 13, thereby to separate ssDNA molecules produced in steps (a), (b), (c), and (d) according to their relative size, each ssDNA comprising an end-label at one or both ends thereof, the apparatus comprising:

(1) electrophoresis means for subjecting the ssDNA to electrophoresis;

(2) electroosmostic flow means for subjecting the ssDNAs to an electroosmostic flow during said electrophoresis;

whereupon subjecting the polymer molecules to simultaneous electrophoresis and electroosmotic flow, the polymer molecules migrate in the solution at different rates, and optionally in different directions, according to their mobility in the solution; and

(3) nucleotide identification means for identifying each nucleotide in a sequence of said DNA molecule according to a mobility of the DNA molecules in the solution.

### BRIEF DESCRIPTION OF THE DRAWINGS

FIG. 1: Peak separation (cm) once the fastest resolved ssDNA (of size M_{c}=100 bases [1]) reaches the detector at L=34 cm, as a function of the number of ssDNA bases (no EOF). Conditions are that of reference [1]: the effective number of uncharged monomers α=α₁Mᵤ is 24, μ₀=1.95×10⁻⁴ cm²/Vs and E=333 V/cm. Inset: The size resolution factor Sₘ for the peaks still inside the capillary when the fastest resolved ssDNA of 100 bases reaches the end of the capillary. This factor, defined in Example 1 (Eq. 8), is the smallest difference in the number of monomers which can be resolved from one another, hence, once the 100 base ssDNA reaches the detector with single monomer resolution (or slightly better), all the smaller molecules inside the capillary are already fully resolved.

FIG. 2: Size resolution factor Sₘ for the various indicated values of \tilde{μ}_{EOF}, as a function of ssDNA size, for the experimental system of reference [1]. The dotted line shows the size resolution factor for negligible EOF conditions; it is clear that \tilde{μ}_{EOF}=1 provides increasingly better resolution (i.e., smaller and smaller differences in the number of monomers can be resolved) with DNA size beyond about 24 ssDNA bases. The predicted read lengths are also indicated and correspond to the intersection of the size resolution factor curves with the horizontal line at Sₘ=1 base.

FIG. 3: Graph of predicted read length as a function of \tilde{μ}_{EOF} (solid line), and the corresponding migration time of the largest resolvable conjugate as a function of \tilde{μ}_{EOF} (dotted line). The horizontal line connects the experimental run time of 18 minutes of reference [1] to the predicted optimal read length without EOF for their conditions of 114 ssDNA bases.

FIG. 4: Graph of size resolution factor Sₘ as a function of number of ssDNA bases, for a scaled EOF mobility of 0.9. The dotted horizontal line at Sₘ=1 indicates the cut-off for single monomer resolution, occurring at about 328 ssDNA bases for this EOF. Conditions are as given in FIG. 1 for reference [1]; the migration length is taken to be 34 cm for both directions of migration. Inset: corresponding migration time in hours; conjugates with more than 115 ssDNA bases require more than 2 hours to reach the detector (horizontal dotted line).

FIG. 5: Graph of predicted read length (solid line) as a function of scaled EOF mobility, for the conditions of reference [1] as given in FIG. 1; the migration length is taken to be 34 cm for both directions of migration. Also shown is the number of ssDNA bases for which the mobility is zero μₑ=μ_{EOF}, i.e. the conjugate size for which the migration time diverges (curve a); between curves b and c, separations take longer than 2 hours.

### DEFINITIONS

‘Drag’—whether used as a noun or as a verb, ‘drag’ refers to impedance of movement of a molecule through a viscous environment (such as an aqueous buffer), such as for example during electrophoresis, either in the presence or the absence of a sieving matrix.

ELFSE—End Labeled Free Solution Electrophoresis. The preferred conditions for ELFSE are apparent to a person of skill in the art upon reading the present disclosure, and the references cited herein

EOF—electroosmotic flow.

‘End label’ or ‘Label’ or ‘tag’ or ‘drag-tag’: refers to any chemical moiety that may be attached to or near to an end of a polymeric compound to increase the drag of the complex during free solution electrophoresis, wherein the drag is caused by hydrodynamic friction. In selected examples, the drag tag may comprise a linear or branched peptide or a polypeptoid comprising up to or more than 300, preferably up to 200, more preferably up to 100 polymer units. Each tag or label may take any form of sufficient configuration or size to cause a sufficient degree of drag during free-solution electrophoresis and/or EOF. For example each label or tag may be a substantially linear, alpha-helical or globular polypeptide comprising any desired amino acid sequence. Moreover, each label or tag may comprise any readily available protein or protein fragment such as an immunoglobulin or fragment thereof, Steptavidin, or other protein generated by recombinant means. In a preferred embodiment each label or tag may be a polypeptoid comprising a linear or branched arrangement of amino acids or other similar units that do not comprise L-amino acids and corresponding peptide bonds normally found in nature. In this way the polypeptoid may exhibit a degree of resistance to degradation under experimental conditions, for example due to the presence of proteinases such as Proteinase K. Preferably, the tags or labels are not charged such that they merely act to cause drag upon the charged polymeric compound during motion through a liquid substance.

MALDI-TOF—matrix-assisted laser desorption/ionization time-of-flight;

‘Near’—In selected embodiments of the invention end labels are described herein as being attached at or near to each end of a polymeric compound. In this context the term ‘near’ refers to attachment of a tag or chemical moiety to a monomeric unit in the vicinity of an end of the polymeric compound, such that the presence of the moiety or tag influences the “end effect” in accordance with the teachings of and discussions of the present application. In addition, the term “near” may vary in accordance with the context of the invention, including the size and nature of the moiety or tag, or the length and shape of the polymeric compound. For example, in the case of a short polynucleotide comprising less than 20 bases, the term “near” may, for example, preferably include those nucleotides within 5 nucleotides from each end of the polynucleotide. However, in the case of a longer polynucleotide comprising more than 100 bases then the term “near” may, for example, include those nucleotides within 20 nucleotides from each end of the polynucleotide.

PEG—poly(ethylene glycol). Typically, “near” can mean within 25%, preferably 15%, more preferably 5% of an end of a polymer molecule relative to an entire length of the polymer molecule;

‘Polymer molecule’—refers to any polymer whether of biological or synthetic origin, that is linear or branched and composed of similar if not identical types of polymer units. In preferred embodiments, the polymer molecules are linear, and in more preferred embodiment the polymeric compounds comprise nucleotides or amino acids. The polymer molecule is preferably a polypeptide or a polynucleotide. More preferably the polymer molecule is a polynucleotide and the method of the present invention is suitable to separate the polynucleotide from other polynucleotides of differing size. Moreover, the polynucleotide may comprise any type of nucleotide units, and therefore may encompass RNA, dsDNA, ssDNA or other polynucleotides. In a more preferred embodiment of the invention, the polymer molecule is ssDNA, and the methods permit the separation of compounds that are identical with the exception that the compounds differ in length by a single nucleotide or a few nucleotides. In this way the methods of the present invention, at least in preferred embodiments, permit the separation and identification of the ssDNA products of DNA sequencing reactions. The size of the tag or label positioned at each end of the ssDNA molecules may (at least in part) be a function of the read length of the DNA sequencing that one may want to achieve. With increasing size of labels or tags the inventors expect the methods of the present invention to be applicable for sequencing reactions wherein a read length of perhaps up to 2000 nucleotides is achieved. With other tags or labels shorter read length may also be achieved including 300, 500, or 1000 base pairs. The desired read lengths will correspond to the use to which the DNA sequencing is applied. For example, analysis such as single nucleotide polymorphism (SNP) analysis may require a read length as small as 100 nucleotides, whereas chromosome walking may require a read length as long as possible, for example up to 2000 base pairs.

‘Polypeptoid’—a linear or non-linear chain of amino-acids that comprises at least one non-natural amino acid that is not generally found in nature. Such non-natural amino acids may include, but are not limited to, D-amino acids, or synthetic L-amino acids that are not normally found in natural proteins. In preferred embodiments, polypeptoids are not generally susceptible to degradation by proteinases such as proteinase K, since they may be unable to form a protease substrate. In selected embodiments, polypeptoids may comprise exclusively non-natural amino acids. In further selected embodiments, polypeptoids may typically but not necessarily form linear or alpha-helical (rather than globular) structures.

‘Preferably’ and ‘preferred’—make reference to aspects or embodiments of the inventions that are preferred over the broadest aspects and embodiments of the invention disclosed herein, unless otherwise stated.

### DETAILED DESCRIPTION OF THE PREFERRED EMBODIMENTS OF THE INVENTION

Polymeric compounds, such as polypeptides and polynucleotides, are routinely subject to modification. Chemical synthesis or enzymatic modification can enable the covalent attachment of artificial moieties to selected units of the polymeric compound. Desirable properties may be conferred by such modification, allowing the polymeric molecules to be manipulated more easily. In the case of DNA, enzymes are commercially available for modifying the 5′ or 3′ ends of a length of ssDNA, for example to phosphorylate or dephosphorylate the DNA. In another example, biotinylated DNA may be formed wherein the biotin moiety is located at or close to an end of the DNA, such that Strepavidin may be bound to the biotin as required. Tags such as fluorescent moieties may also be attached to polynucleotides for the purposes of conducting DNA sequencing, for example using an ABI Prism™ sequencer or other equivalent sequencing apparatus that utilizes fluorimetric analysis

End Labeled Free Solution Electrophoresis (ELFSE) provides a means of separating DNA with free solution capillary electrophoresis, eliminating the need for gels and polymer solutions which increase the run-time and can be difficult to load into a capillary. In free solution electrophoresis, DNA is normally free-draining and all fragments reach the detector at the same time, whereas ELFSE uses an uncharged label molecule attached to each DNA fragment in order to render the electrophoretic mobility size-dependent. With ELFSE, however, the larger molecules are sometimes not sufficiently separated (limiting the read length in the case of ssDNA sequencing) while the smaller ones are sometimes over-separated; the larger ones are too fast while the shorter ones are too slow, which is the opposite of traditional gel-based methods. In this application, the inventors show how an electroosmotic flow can be used to overcome these problems and extend the DNA sequencing read length of ELFSE. This counter-flow allows the larger, previously unresolved molecules more time to separate, thereby increasing the read length. Through careful investigation, the inventors show that an electroosmotic flow mobility of approximately the same magnitude as that of unlabeled DNA would provide the best results for the regime where all molecules move in the same direction. Even better resolution would be possible for smaller values of electroosmotic flow which allow different directions of migration; however the migration times might become too large. The flow should preferably be well controlled since the gain in read length decreases as the magnitude of the counter-flow increases; an electroosmotic flow mobility double that of unlabeled DNA would no longer increase the read length, although ELFSE would still benefit from a reduction in migration time.

End labeled free solution electrophoresis (ELFSE) is a relatively new technique that achieves separation of various lengths of DNA in free solution [1, 2, 3, 4]. This is accomplished by attaching an uncharged (or nearly so) end label called a drag molecule (or drag-tag) of a set size to each DNA fragment in order to render the resulting conjugate's electrophoretic mobility length-dependent, and overcome the free-draining phenomenon which normally leads to co-migration of all lengths of DNA in free solution (except very small fragments [5, 6]) [7, 8, 9, 10]. This phenomenon is the reason why most DNA separations are performed in a gel which selectively slows down longer polymers more by forcing them to collide more frequently with gel fibers [11]). The key to separation by the ELFSE technique lies in the drag-tag adding a set resistance (friction) to the motion of each DNA fragment, meaning that the more charged monomers a conjugate has (i.e., the longer the DNA component), the more force it has to pull the drag-tag. Hence larger conjugates go faster and vice versa, leading to size-based separation in free solution. Ren et al. [1] have successfully used this technique to sequence up to about 100 base long ssDNA molecules in about 18 minutes in a 34 cm long capillary; their drag-tag was the globular protein streptavidin.

The theory generally used to analyze ELFSE data indicates that the electrophoretic mobility μₑ of an undeformed conjugate molecule comprising M_{c} charged monomers (e.g., the number of ssDNA bases in the case of DNA sequencing) and Mₙ uncharged monomers (the drag-tag) is given in references [2-4, 12, 13]:

$${μ}_{e} = {μ}_{0} \frac{{M}_{c}}{{M}_{c} + {α}_{1} {M}_{u}} \qquad ( 1 )$$

where μ₀ is the length-independent free solution mobility of unconjugated ssDNA. This equation, based on the work of Long et al. [14], has been shown to provide good fits to experimental data [2]. The α₁ value is a microscopic constant which accounts for the difference in monomer size and stiffness between the uncharged and charged monomers such that the product α=α₁Mᵤ is the number of charged ssDNA monomers that have the same friction coefficient as the drag-tag, yielding a total number of effective monomers (each having the same friction coefficient) in the conjugate of M=M_{c}+α₁Mᵤ. For example, the streptavidin drag-tag tested for ssDNA sequencing with ELFSE has an effective friction parameter α=α₁Mᵤ≅24−40, depending on the ionic strength of the buffer [1]. (Note that the calculations in [1] need to be adjusted to take into account recent improvements to ELFSE theory [2, 4]; however, the α=α₁Mᵤ value can be taken directly from the slope of their fit in FIG. 7). The net mobility of the conjugate given by Eq. 1 is simply a uniformly weighted average of the individual effective monomer mobilities. The migration time

$$t = \frac{L}{μ E} ,$$

i.e. the time taken by the analyte to travel the distance L to the detector, is thus given by:

$$t = \frac{L}{{μ}_{0} E} \times \frac{{M}_{c} + {α}_{1} {M}_{u}}{{M}_{c}} = {t}_{0} \times ( 1 + \frac{{α}_{1} {M}_{u}}{{M}_{c}} ) \qquad ( 2 )$$

where E is the electric field strength and

$${t}_{0} = \frac{L}{{μ}_{0} E}$$

is the migration time of an unlabelled ssDNA fragment. The temporal peak spacing can be obtained by taking the derivative of the migration time with respect to the number of charged monomers since there is one peak per charged segment length:

$$\vert \frac{\partial t}{\partial {M}_{c}} \vert = \frac{\vert {t}_{0} - t \vert}{{M}_{c}} ∼ \frac{1}{{M}_{c}^{2}} . \qquad ( 3 )$$

One can see that the peak spacing decreases very quickly with M_{c}; hence conjugates with larger ssDNA fragments, the fastest ones, have very small peak spacing (although they also form very narrow peaks because their short migration times and large molecular weights minimize diffusional peak broadening). As a result, longer ssDNA have peaks that overlap and are less resolved with ELFSE; this process appears to be what limits the read length (currently, about 100 bases can be sequenced with streptavidin without any special base calling software [1]). This is the major issue to overcome in order for ELFSE to become competitive with other DNA sequencing techniques. The read length would obviously increase if the peak spacing (Eq. 3) could be increased for the longer ssDNA.

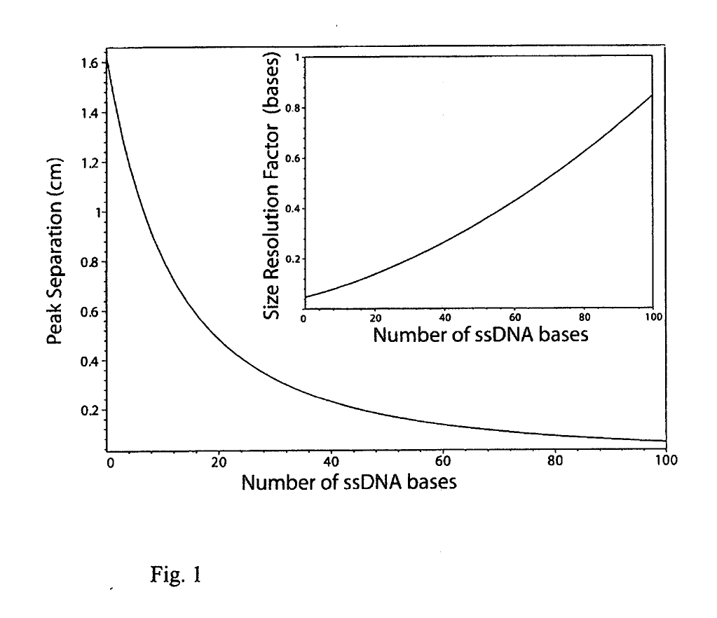

Remarkably, unlike most electrophoresis systems, once the fastest resolved molecules reach the detector with ELFSE, all of the slower conjugates are already separated in the channel. In the case of reference [1] for instance, the smallest molecules (starting at about 23 bases long, including the primer size) took about 18 minutes to reach the detector but they were already resolved by the time the largest resolved molecule (about 100 ssDNA bases) reached the detector at t≈10 min. The results presented in the experimental article of reference [1] throughout this application in order to illustrate the invention. The predicted peak spacing of all the smaller ssDNA molecules still in the capillary when the largest resolved conjugate (M_{c}′) reaches the detector is shown in FIG. 1. The position of all the smaller molecules when the largest DNA resolved by reference [1] (M_{c}′=100 ssDNA bases) reached the detector at t(M_{c}′)≈10ₘᵢₙ, x(M_{c})=μₑ(M_{c})×E×t(M_{c}′), was calculated through use of Eq. 1 and the values of α=24 bases, μ₀=1.95×10⁻⁴ cm²/Vs and E=333 V/cm given by these authors. The derivative with respect to M_{c} of the position gives the spatial peak spacing. From FIG. 1, it is clear that all of the smaller ssDNA have a much greater peak spacing at the time of detection of the fastest resolved ssDNA; this is the reason why [1] observed smaller molecule peaks that were needlessly over-separated by the time they reached the detector. The inset of FIG. 1 shows the corresponding predicted size resolution factor Sₘ (defined in the next section, Eq. 8), which is the smallest difference in the number of monomers which can be resolved from one another. Clearly a better than single monomer resolution (Sₘ≦1 monomer, as needed for sequencing) is achieved for all the remaining peaks once the largest (100 ssDNA bases) resolved conjugate reaches the detector; in fact the resolution is even better for smaller conjugates. Since the size resolution factor Sₘ is lower for all the conjugates still in the capillary once the largest resolved molecule reaches the detector, a whole-capillary snapshot detection mode would immediately yield an electropherogram with single monomer resolution (or better) for M_{c}=0 through M_{c}=M_{c}′=100 bases in this case. However, with the usual finish line detection mode employed with capillary electrophoresis, one must wait for the slower (smaller) molecules to reach the detector at the end of the capillary by which time they are needlessly over-separated.

With traditional ELFSE the longest conjugates are not separated enough to be resolved, while the shorter ones are over-separated; the longer ones are too fast while the shorter ones are too slow, the opposite of the situation with regular electrophoresis performed in a gel or polymer solution. In order to slow down the longer conjugates and allow them more time to separate, and to speed up the smaller conjugates, the inventors perform ELFSE in the presence of an electroosmotic flow (EOF). This counter-flow, which is constant [15] (assuming that the capillary is uniformly charged and both ends are at the same pressure [16]), arises as a consequence of the negative charges of the uncoated inner capillary wall surface, and results in the analyte motion proceeding in the reverse direction. In the presence of EOF, the conjugates are carried along by the opposing flow, resisting the motion to an extent determined by their own electrophoretic mobility μₑ. Hence the fastest (longest) conjugates in traditional ELFSE would become the slowest in the presence of EOF since they could fight this flow the most, and vice versa.

In order to increase the read length, the peak spacing given by Eq. 3 needs to be increased for larger molecules, for which the numerator |t₀−t| (i.e., the absolute difference in migration time between unlabeled and labeled DNA) is almost zero because very large ssDNA fragments can pull the drag-tag with ease and approach the speed of unlabeled ssDNA. There are four ways to increase the numerator. Most simply, a) a longer capillary and/or b) a lower electric field strength could be used to increase both the migration times t and t₀, and thereby increase their absolute difference (actually the former will increase the peak spacing for most electrophoretic systems, including gel based methods, however with the latter the gain in peak spacing may unfortunately be accompanied by an insurmountable increase in diffusion). Another means of increasing the numerator is to c) use a drag-tag capable of exerting greater frictional drag which would decrease t while leaving t₀ unaffected (in fact increasing the frictional properties of the drag-tag is a main goal of current ELFSE research; however, it is extremely challenging experimentally [4]). Finally, while Eq. 3 would need to be adjusted for the presence of EOF, one would expect intuitively that if d) the EOF were properly chosen it could increase both t and t₀, leading to an increase in peak spacing by slowing down both unlabeled and labeled ssDNA. Thus the EOF may indeed increase the read length of ELFSE; furthermore, it may also reduce the unnecessary over-separation of small conjugates.

The following examples illustrate and describe preferred embodiments of the invention, and are in no way intended to be limiting with respect to the invention disclosed and claimed herein.

### EXAMPLES

### Example 1

ELFSE in the Presence of EOF

In this example the inventors develop detailed equations governing ELFSE in the presence of EOF, and investigate the predicted electrophoretic behaviour. As previously mentioned, the EOF is assumed to simply add a constant term μ_{EOF} to the electrophoretic mobility of the analyte. The EOF results from the negative charges on the inner surface of uncoated fused silica capillary walls which attract positive ions from solution. While the negative charges of the wall are immobile, the positive charges of the thin Debye layer (typically 1-10 nm [16]) neighbouring the surface are free to move and hence once an electric field is applied, they move towards the cathode. Their motion drags the fluid from the bulk solution along with them, creating the plug-like electroosmotic flow. This flow is generally constant and in the opposite direction to the ssDNA conjugate's own mobility μₑ, such that the net mobility of the analyte is the difference of these two mobilities [16]:
μ=μ_{EOF}−μₑ (4)
where μₑ is the mobility of the analyte under conditions of no EOF, as given in Eq. 1. The magnitude of the EOF mobility μ_{EOF} depends on the extent and character of the capillary wall coating; a bare wall exhibits the highest EOF mobility. Whenever the proper mobility μₑ of the analyte is exceeded by the mobility due to the electroosmotic flow μ_{EOF}, the migration proceeds in the opposite direction, with the conjugate moving towards the cathode instead of the anode. The net migration time in the presence of EOF

$$t = \frac{L}{\vert μ \vert E} ,$$

is thus given by:

$$t = \frac{L}{{μ}_{0} E} ( \frac{1}{\vert {\tilde{μ}}_{EOF} - {\tilde{μ}}_{e} \vert} ) \qquad ( 5 )$$

where the dimensionless mobility ratios \tilde{μ}_{EOF} and \tilde{μ}ₑ are defined as follows:

$$\begin{matrix}{\tilde{μ}}_{EOF} ≡ \frac{{μ}_{EOF}}{{μ}_{0}} & ( 6 ) \\ {\tilde{μ}}_{e} ≡ \frac{{μ}_{e}}{{μ}_{0}} = \frac{{M}_{c}}{{M}_{c} + {α}_{1} {M}_{u}} . & ( 7 )\end{matrix}$$

Since the conjugate's proper mobility decreases due to the drag molecule of effective hydrodynamic size α=α₁Mᵤ (i.e. μₑ≦μ₀), the maximum proper mobility of a conjugate is μ₀, and a scaled EOF mobility \tilde{μ}_{EOF} exceeding 1 means that all of the conjugates migrate in the opposite direction in the presence of the electroosmotic flow. The inventors first investigate this case where all conjugates travel in the same direction, i.e., scaled EOF mobilities in the range \tilde{μ}_{EOF}≦1, and then the case for \tilde{μ}_{EOF}≦1. Under the former conditions, the conjugates which were the fastest in the traditional EOF-free direction become the slowest in the opposite direction because they can fight the flow the hardest, and vice versa, as previously mentioned. Remarkably, the inventors note that for \tilde{μ}_{EOF}=1, the temporal peak spacing |∂t/∂M_{c}| is constant (as can be verified by taking the derivative of Eq. 5 with respect to M_{c}), whereas it decreases with increasing ssDNA size M_{c} (similar to all other separation methods) for any other value of \tilde{μ}_{EOF}≧1.

The viability of ELFSE separations in the presence of EOF was shown by Heller et al. [10] for double-stranded DNA, although with apparently less success than without the EOF. In the following the inventors investigate how ELFSE separations are affected by the EOF, and in particular how they depend on the scaled EOF mobility \tilde{μ}_{EOF}. The inventors define the size resolution factor as the ratio of the temporal full width at half maximum FWHMₜ, to the temporal peak spacing |∂t/∂M_{c}| as the bands pass in front of the detector:

$${S}_{m} ( {M}_{c} , {\tilde{μ}}_{EOF} ) = \frac{{FWHM}_{t}}{\vert \frac{\partial t}{\partial {M}_{c}} \vert} \qquad ( 8 )$$

where the units of Sₘ are number of monomers. This factor represents the smallest difference in the number of monomers which can be resolved from one another. An Sₘ(M_{c},\tilde{μ}_{EOF}) factor of 1 (i.e., single monomer resolution) or less is hence necessary for sequencing; clearly, smaller values of this factor correspond to an increase in the resolution power of the system. Following the development in [4, 13], this factor can be expressed as follows for the electrophoretic system of reference [1] (see Appendix A for a brief derivation):

$${S}_{m} ( {M}_{c} , {\tilde{μ}}_{EOF} ) \approx \frac{{( \vert {\tilde{μ}}_{EOF} - {\tilde{μ}}_{e} \vert )}^{1 / 2} {( {M}_{c} + 24 )}^{7 / 4}}{5088} . \qquad ( 9 )$$

The development of this equation assumes that the conjugates are in a Gaussian coil conformation, that the drag-tags are completely monodisperse, and that the band loading width is negligible. The inventors take into account only thermal (diffusion) band broadening (as is the case for experimentally optimal conditions), and neglect any additional band broadening which may arise due to the EOF (for non-ideal effects, see [16-18]). The predictions compare well with the experimental results of reference [1]. For instance, the inventors note that the predicted size resolution factor for the largest resolved ssDNA as shown in the inset of FIG. 1 is slightly less than 1, indicating that even better resolution could be expected were their experimental conditions ideal; however, the initial loading width was not completely negligible, there may have been some other sources of non-thermal band broadening, and the label was slightly polydisperse. Nonetheless, their experimental results are very close to what the inventors expect based on ideal conditions, indicating that their conditions were close to optimized, and reinforcing the importance of the invention.

### Example 2

Electroosmotic Flow Mobility Exceeding the Mobility of All Conjugates: Single Direction of Migration

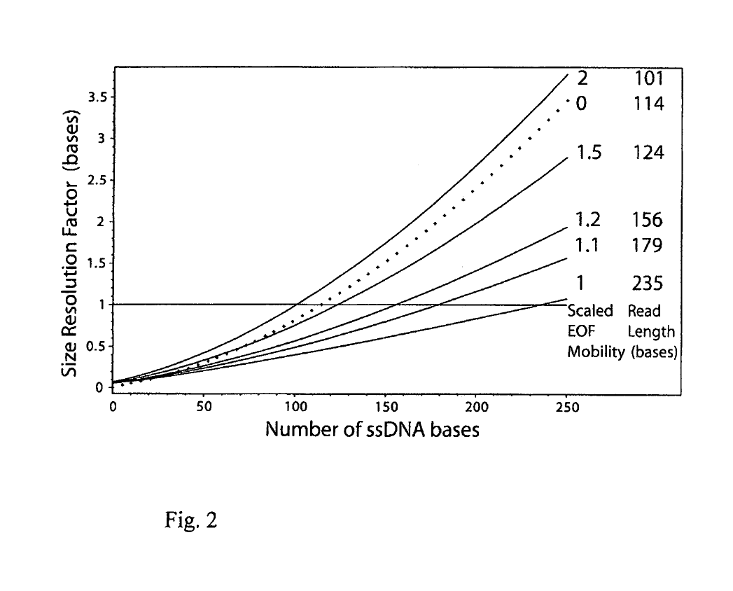

Here, the inventors investigate ELFSE in the presence of an EOF mobility μ_{EOF} that exceeds the DNA conjugates own proper mobility μₑ (i.e. the mobility that it would have in the absence of EOF) which has a maximal value of the mobility of unlabeled DNA μ₀; hence we are looking at the situation \tilde{μ}_{EOF}≧1 where all conjugates travel backwards, carried by the EOF. The predicted size resolution factor Sₘ(M_{c},\tilde{μ}_{EOF}) using the experimental parameters of reference [1] is plotted in FIG. 2 for various values of \tilde{μ}_{EOF}≧1. One can see clearly that scaled EOF mobilities \tilde{μ}_{EOF} close to 1 would provide increasingly better size resolution with DNA size (i.e., smaller and smaller differences in the number of monomers could be resolved) than the same experimental system with no EOF; for \tilde{μ}_{EOF}=1, this improvement is expected to begin at about 24 ssDNA bases. As the ratio \tilde{μ}_{EOF} increases from 1 to 2, the expected gain in resolution quickly decreases; indeed for \tilde{μ}_{EOF}=2 the curve lies above that for negligible EOF, indicating that slightly poorer resolution would be achieved than with negligible EOF conditions.

For each curve in FIG. 2 the predicted read length is shown, and corresponds to the intersection of the curve with the horizontal line at Sₘ=1 which represents the cut-off for single-monomer resolution. These predictions were obtained by setting Eq. 9 equal to one and solving numerically for M_{c}; i.e., since S₅(M_{c},\tilde{μ}_{EOF}) is the number of monomers that can be resolved, and since this value strictly increases with the number of ssDNA bases M_{c} (for all values of \tilde{μ}_{EOF}≧1), setting it equal to one gives the largest ssDNA that can be resolved on a single monomer basis. Note that ssDNA sequences can often be determined even when peaks are not completely resolved, especially with the aid of sophisticated base calling software, and hence the predicted read lengths are essentially lower bounds for experimentally ideal (diffusion limited) conditions. While for negligible EOF, the read length for the experimental system of reference [1] is predicted to be about 114 bases (as discussed above, this is similar to the value obtained experimentally of about 100 bases [1], and likely differs due to somewhat non-ideal experimental conditions such as non-negligible initial loading width), for \tilde{μ}_{EOF}=1 it is expected to be much higher, at about 235 bases. Hence an EOF with \tilde{μ}_{EOF}=1 would provide substantially better performance than conditions of negligible EOF, extending the read length by over 200 percent. The read length predicted for \tilde{μ}_{EOF}=1.1 is still a good improvement over the EOF-free case, at about 179 bases. Although the improvement to the resolution quickly drops off as the mobility \tilde{μ}_{EOF} increases from 1, the read length for \tilde{μ}_{EOF}=1.5 is still expected to be better than that of the EOF-free case, at 124 bases. Once the mobility \tilde{μ}_{EOF} reaches 1.65, the read length returns to that obtained without EOF, however as will be demonstrated next, using the EOF still offers the advantage of lower total run times.

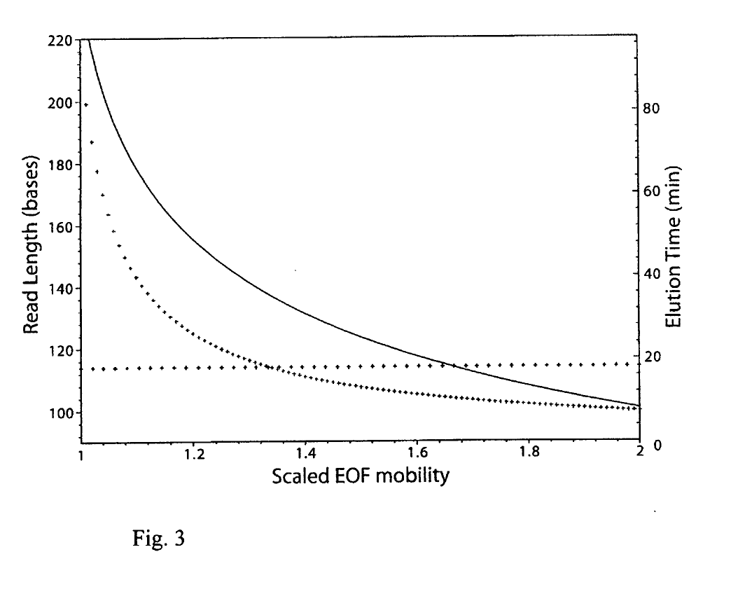

FIG. 3 shows the predicted read length as a function of \tilde{μ}_{EOF}≧1 for the experimental system of reference [1]; the corresponding migration time for the largest resolvable molecules is also shown. Under ideal conditions the inventors predict that without the EOF the read length would be 114 bases; this could also be achieved with \tilde{μ}_{EOF}=1.66, which would have a corresponding run time of only 10.5 minutes, compared to the 18 minutes required without EOF, as found experimentally by reference [1] (note that this is about the run time the inventors would expect were a snap-shot detection mode available for capillary electrophoresis without EOF, see Introduction). The horizontal line connects this experimental run time of 18 minutes to the optimized negligible-EOF read length prediction of 114 bases. As can be seen from FIG. 3, running ELFSE in the presence of EOF allows not only for a substantial increase in the read length, it also shortens the total run time for all values of scaled EOF mobility \tilde{μ}_{EOF}≧1.34. For values of scaled EOF mobility 1≦\tilde{μ}_{EOF}≦1.34, more time is required for the resulting increase in read length. For \tilde{μ}_{EOF}≧1.66 the separations have a shorter read length with the EOF, but take less time. In the intermediate regime 1.34≦\tilde{μ}_{EOF}≦1.66, increased read length is accompanied by shorter migration times.

### Example 3

Electroosmotic Flow Mobility Less Than the Mobility of the Fastest Conjugate: Two Migration Directions

In this section the inventors look at the situation where the EOF is small enough (\tilde{μ}_{EOF}≦1) that some of the faster conjugates can fight it and migrate forwards, in the same direction as they would in the absence of EOF. Hence there are smaller molecules moving backwards and larger molecules that are fast enough to overcome the EOF moving forwards. In order to detect both sets of molecules, one would require a different experimental set up, such as injection in the middle of the capillary with detection occurring at both ends, or using multiple runs each geared for a specific size range (and direction). For simplicity take the length L from injection to the detector to be the same for both sets of molecules (although different migration lengths might improve the throughput). An EOF mobility μ_{EOF} slightly less than that of unlabeled DNA μ₀ would be even closer to the mobility of very long DNA μₑ (which is slightly less than p due to the presence of the label) than it would be for μ_{EOF}=μ₀. Therefore the longer conjugates would be given even more time to separate from each other; thereby further increasing the read length.

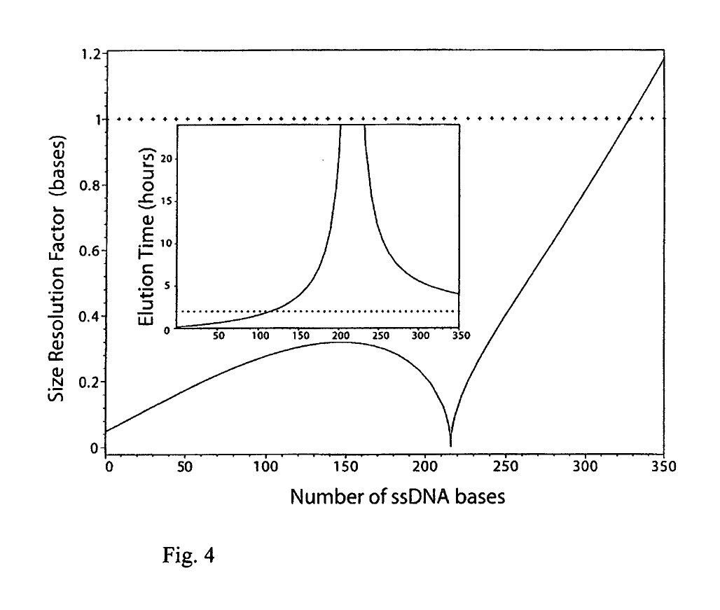

FIG. 4 shows the size resolution factor Sₘ as a function of the number of ssDNA bases for a scaled EOF mobility of 0.9, for the conditions of reference [1] as given in FIG. 1, taking the migration length to be 34 cm for both directions of migration. The dotted horizontal line at Sₘ=1 indicates the cut-off for single monomer resolution and gives a read length of 328 ssDNA bases for this EOF. The minimum in the curve occurring at 216 ssDNA bases corresponds to the size of DNA which has a mobility approximately equal to the EOF mobility. Hence for a scaled EOF mobility of 0.9, molecules having more than 216 ssDNA bases can fight the EOF and move forwards, and molecules less than this size are carried backwards by the EOF, while the conjugate with 216 ssDNA bases barely moves. The best resolution occurs near this minimum. Since all of the conjugates with less than 216 bases have a size resolution factor less than 1, they are all resolved, while larger and larger molecules have lesser resolution (due to their ability to fight the EOF and attain speeds that do not allow for adequate separation), eventually reaching that of single monomer resolution for 328 bases. This read length is excellent for ELFSE; unfortunately, for DNA sizes near the size resolution factor minimum, i.e. those that barely move because their own proper mobility is almost the same as the EOF, the migration time would be very long, in fact the migration time diverges. The inset of FIG. 4 shows the corresponding migration time for the scaled EOF mobility of 0.9. Conjugates with more than 115 ssDNA bases require more than 2 hours to reach the detector, while those molecules with mobilities approximately equal to that of the EOF, i.e., those having 216 plus or minus a few ssDNA bases, barely move and hence do not reach the detector in any reasonable amount of time.

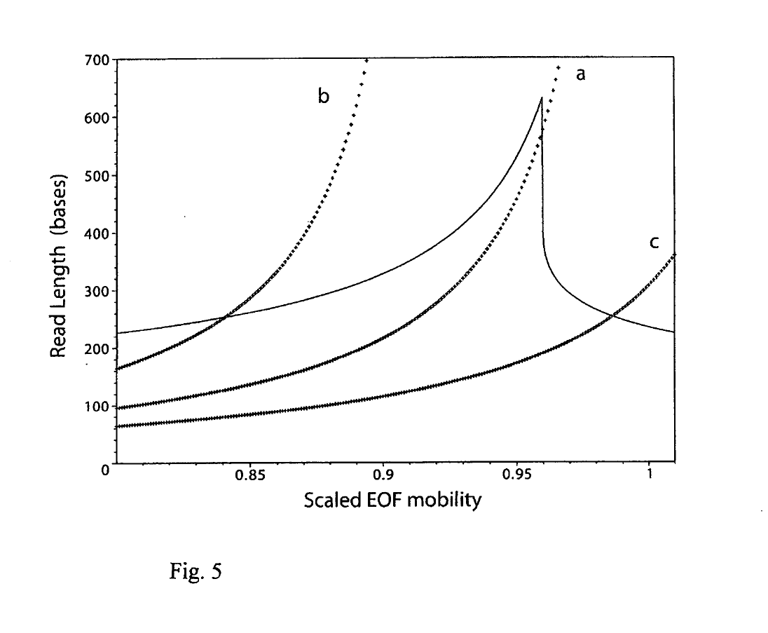

FIG. 5 shows the predicted read length as a function of \tilde{μ}_{EOF} for the conditions of reference [1] as given in FIG. 1. Also shown is the number of ssDNA bases for which the mobility is zero (μₑ=μ_{EOF}), i.e. the conjugate size for which the migration time diverges (curve a); between curves b and c, separations take longer than 2 hours. Unfortunately, although the read lengths are exceptional for 0.9\tilde{<}\tilde{μ}_{EOF}\tilde{<}0.96, separations in this EOF range require too much time and are likely only of interest for special applications. A scaled EOF mobility of 0.99 would still allow for all conjugates (under the conditions of reference [1]) to move in the same direction while slightly increasing the read length over that of \tilde{μ}_{EOF}=1 (235 ssDNA bases) to 248 ssDNA bases, with a corresponding increase in migration time from 1.6 hours to 1.9 hours. It should also be noted that even if the curves are resolved, for these long migration times the bands will also be fairly wide and will take some time to pass in front of the detector. For example, with a scaled EOF mobility of 0.99, under the conditions of reference [1], the slowest band (i.e. the largest ssDNA) would take about 28 seconds to pass in front of the detector; it is possible that for these spread out bands, the signal to noise ratio may be too low to detect.

Since the migration time becomes a limiting factor for the read length, systems which shorten the run time would increase the gains expected through use of EOF for ELFSE. All of the discussions presented are based on the capillary electrophoretic system of reference [1]; with the increased speed of microchip electrophoretic systems even better gains due to the EOF could be expected by overcoming the time restraints. The data presented here could be easily adapted for such systems which may indeed make EOF-based ELFSE a competitive sequencing technique, allowing for rapid, high read length separations void of the need for gels or entangled polymer solutions.

### Example 4

Review

The inventors have shown that the EOF can be used to dramatically extend the read length of DNA separations by ELFSE by improving the resolution of larger molecules. For the case of all molecules migrating in the same direction (i.e., \tilde{μ}_{EOF}≡μ_{EOF}/μ₀≧1), the best resolution is expected when the scaled EOF mobility is near unity, and positive effects drop quickly with an increase in \tilde{μ}_{EOF}. For example, a scaled EOF mobility of unity could more than double the read length for the system of reference [1] (for which optimal conditions would be expected to yield a read length of 114 ssDNA bases without the EOF), extending it to 235 ssDNA bases. For the case of smaller molecules migrating backwards with the EOF and larger molecules moving forwards against the EOF (i.e., \tilde{μ}_{EOF}≦1), even more exceptional improvements to the read length are expected; however the long run time makes this useful for special applications only. For the conditions of reference [1], a scaled EOF mobility of 0.99 would still allow all the molecules to migrate in the same direction, and the read length is predicted to be 248 ssDNA bases, an exceptional improvement over the predicted optimal read length of 114 bases for ELFSE without EOF.

In order to take advantage of the EOF based resolution increase, the exact value of the scaled EOF mobility is preferably well controlled. The coating on the capillary wall surface is a key factor determining EOF. Heller et al. [10] reduced the EOF from that of an uncoated capillary by 50%, to 1×10⁻³ cm²/Vs through use of a thin polyacrylamide coating. This corresponds to a scaled BOF mobility in the range 2<\tilde{μ}_{EOF}<10, given that values of μ₀ typically range from 1×10⁻⁴ cm²/Vs to 5×10⁻⁴ cm²/Vs. Hence the EOF would typically need to be reduced by 75% or more in order to achieve a \tilde{μ}_{EOF} value near unity, for example. Another means of controlling the EOF is by the application of an external electric field which forms a potential gradient with the usual internal electric field thereby creating a radial field; this adjustable gradient is perpendicular to the capillary wall and changes the density of electric charge on the inner capillary wall, thereby allowing for control of the EOF [19-21]. In addition to the EOF, all factors influencing the mobility would also need to be well controlled so as to maintain a constant μ₀ since the desired EOF mobility depends upon this value.

In addition to the clear resolution advantage of performing ELFSE in the presence of EOF, the decrease in run-time would also be a big benefit; indeed even non-optimal EOF values (\tilde{μ}_{EOF}≧1.34) which would not substantially improve resolution, would still shorten the total time required for the electropherogram. For values of scaled EOF mobility 1≦\tilde{μ}_{EOF}≦1.34, more time is required for the resulting increase in read length. The EOF would also change the order of detection as the smaller conjugates reach the detector first, followed by the larger conjugates, restoring the usual order, as with standard (gel/entangled polymer) sequencing, and eliminating the unnecessary wait for small, already resolved molecules to travel to the detector. If the EOF could be maintained at \tilde{μ}_{EOF}=1, one could also expect evenly spaced peaks which may allow for easier base calling algorithms; somewhat larger values of \tilde{μ}_{EOF} would give approximately constant peak spacing which would also be beneficial for base calling.

In order to achieve comparable read lengths without EOF, very powerful voltage supplies would be necessary. For example, to obtain a read length comparable to the 235 bases predicted with \tilde{μ}_{EOF}=1 without the EOF (for the system of reference [1]), one would need a 3.3 m long capillary which would require a much greater voltage in order to maintain the electric field strength at approximately 333 V/cm. Similarly, comparable read lengths obtained via an increase in the electric field would require an electric field strength of about 3300 V/cm, which would also be very demanding indeed in terms of the power supply source. Not only would the field strengths required be extreme, but they might also be accompanied by an unfavourable increase in peak widths. Using the electroosmotic flow is a powerful alternative to these extreme and unrealistic approaches.

The inventors also note that while one could use a method other than the EOF to create the counter flow in an attempt to take advantage of the potential gains, such as a pressure difference, it would lack the characteristic EOF plug-like flow. Typically, non-EOF based counter flows have a parabolic profile, in contrast to the flat profile across the bulk fluid obtained with EOF. It is only with a flat profile that all molecules across the diameter of the capillary experience the same rate of counter flow; a parabolic profile would mean that molecules near the center would be subject to a greater counter flow than those closer to the outside, leading to an undesirable band broadening.

The present discussion of ELFSE behaviour in the presence of EOF is based on negligible band loading width and assume that any EOF-based band broadening effects are negligible. For systems where this assumption is not entirely justified, adjustments may need to be made. It is important to note as well that the drag molecule for ELFSE in the presence of EOF would need to be free of problems of sticking to the uncoated (or less coated) capillary wall.

### Example 5

A Brief Derivation of Eq. 9

In the following the inventors provide a brief derivation of Eq. 9, the size resolution factor for the system of reference [1]. The definition of this factor is given by Eq. 8. First, we start with the numerator, the temporal full width at half maximum (assuming Gaussian peaks):
FWHMₜ=2\sqrt{21n(2)}σₜ (10)
where σₜ is the temporal standard-deviation and can be given as follows when the initial peak width is negligible and diffusion is the only significant source of band broadening:

$${σ}_{t} = \frac{\sqrt{2 D t}}{v} \qquad ( 11 )$$

where v=L/t is the velocity, D=k_{B}^{T}/4πηR_{G} is the Zimm diffusion coefficient of the hybrid ssDNA molecule, k_{B} is the Boltzmann constant [13], T is the absolute temperature, η is the viscosity of the free solution and R_{G} is the radius of gyration. Hence the numerator of Eq. 8 can be rewritten as follows, where v has been replaced by L/t:

$${FWHM}_{t} = \frac{2}{L} \sqrt{\frac{ln ( 2 ) {k}_{B} T}{π η {R}_{G}}} \times {t}^{3 / 2} . \qquad ( 12 )$$

Following the blob approach presented in [4, 13] which rescales the charged and uncharged segments to account for their different hydrodynamic sizes, the total radius of gyration of the conjugate molecule can be given by that of its charged (R_{G}_{_{c}}) and uncharged segments (R_{G}_{ₙ}):
R_{G}={square root over (R_{G}_{_{c}}²R_{G}_{ₙ}²)}. (13)
If one assumes excluded volume effects to be negligible, the radii of gyration are given by:

$${R}_{{G}_{i}} = \sqrt{\frac{{b}_{{K}_{i}} {b}_{i} {M}_{i}}{6}} \qquad ( 14 )$$

where b_{K}_{ᵢ} is the Kuhn length of polymer i, a measure of its stiffness, and bᵢ is the monomer size of polymer i. Hence the total radius of gyration of the conjugate can be written as:

$${R}_{G} = \sqrt{\frac{{b}_{{K}_{c}} {b}_{c} {M}_{c}}{6} + \frac{{b}_{{K}_{u}} {b}_{u} {M}_{u}}{6}} = \sqrt{\frac{{b}_{{K}_{c}} {b}_{c}}{6} \times ( {M}_{c} + {α}_{1} {M}_{u} )} \qquad ( 15 )$$

where

$$α ≡ \frac{{b}_{{K}_{u}} {b}_{u}}{{b}_{{K}_{c}} {b}_{c}} ,$$

as given by reference [4, 13]. Using Eqs. 5 and 7 for the denominator of the size resolution factor, one finds:

$$\vert \frac{\partial t}{\partial {M}_{c}} \vert = \frac{{α}_{1} {M}_{u} t}{( {\tilde{μ}}_{EOF} - {\tilde{μ}}_{e} ) {( {M}_{c} + {α}_{1} {M}_{u} )}^{2}} \qquad ( 16 )$$

Substituting Eq. 15 into the expression for the numerator, Eq. 12, and using Eq. 16 for the denominator, the size resolution factor becomes:

$${S}_{m} \approx \frac{2}{L} \sqrt{\frac{ln ( 2 ) {k}_{B} T}{π η \sqrt{\frac{{b}_{{K}_{c}} {b}_{c}}{6}}}} \times \frac{( \vert {\tilde{μ}}_{EOF} - {\tilde{μ}}_{e} \vert ) {( {M}_{c} + {α}_{1} {M}_{u} )}^{7 / 4} {t}^{1 / 2}}{{α}_{1} {M}_{u}} . \qquad ( 17 )$$

Again making use of Eq. 5 for the migration time, we find:

$${S}_{m} \approx 4 \sqrt{\frac{ln ( 2 ) {D}_{0}}{L {μ}_{0} E}} \times \frac{{( \vert {\tilde{μ}}_{EOF} - {\tilde{μ}}_{e} \vert )}^{1 / 2} {( {M}_{c} + {α}_{1} {M}_{u} )}^{7 / 4}}{{α}_{1} {M}_{u}} \qquad ( 18 )$$

where the constant D₀ is defined by

$${D}_{0} ≡ \frac{{k}_{B} T}{4 π η \sqrt{\frac{{b}_{{K}_{c}} {b}_{c}}{6}}} D \sqrt{{M}_{c} + {α}_{1} {M}_{u}} \qquad ( 19 )$$

and can be found from the Ren et. al. [1] value of the diffusion coefficient D=4.8×10⁻⁷ cm²/s, reported for M_{c}=61 bases and α₁Mᵤ=24 bases to be D₀=4.43×10⁻⁶ cm²/s. Using this value, along with the experimental values from [1] presented above (L=34 cm, α₁Mᵤ=24, μ₀=1.95×10⁻⁴ cm²/Vs and E=333 V/cm) we arrive at Eq. 9 for the size resolution factor for the experimental conditions of reference [1]:

$${S}_{m} ( {M}_{c} , {\tilde{μ}}_{EOF} ) \approx \frac{{( \vert {\tilde{μ}}_{EOF} - {\tilde{μ}}_{e} \vert )}^{1 / 2} {( {M}_{c} + 24 )}^{7 / 4}}{5088} . \qquad ( 9 )$$

The general equation for the ELFSE size resolution factor for charged-uncharged conjugates solely experiencing thermal-based diffusion is:

$${S}_{m} \approx 4 \sqrt{\frac{ln ( 2 ) D}{L {μ}_{0} E}} \times \frac{{( \vert {\tilde{μ}}_{EOF} - {\tilde{μ}}_{e} \vert )}^{1 / 2} {( {M}_{c} + {α}_{1} {M}_{u} )}^{2}}{{α}_{1} {M}_{u}} . \qquad ( 20 )$$

Since the inventors expect the mobilities to be independent of electric field, it can be seen that it is the total voltage drop, i.e. the factor E×L, that determines the resolution rather than either the electric field strength or the migration length independently. Also the inventors see that the viscosity η of the electrophoresis medium does not affect the size resolution of the system (η cancels out in the ratio D/μ₀).

While the invention has been described with reference to particular preferred embodiments thereof, it will be apparent to those skilled in the art upon a reading and understanding of the foregoing that numerous methods for polymer molecule modification and separation, as well as corresponding apparatuses for their separation, other than the specific embodiments illustrated are attainable, which nonetheless lie within the spirit and scope of the present invention. It is intended to include all such methods and apparatuses, and equivalents thereof within the scope of the appended claims.

### REFERENCES

1 Ren, H., Karger, A. E., Oaks, F., Menchen, S., Slater, G. W., Drouin, G., Electrophoresis 1999, 20,2501-2509.
2 Desruisseaux, C., Long, D., Drouin, G., Slater, G. W., Macromolecules 2001,34, 44-52.
3 Desruisseaux, C., Drouin, G., Slater, G. W., Macromolecules 2001, 34, 5280-5286.
4 Meagher, R. J., Won, J.-I., McCormick, L. C., Nedelcu, S., Bertrand, M. M., Bertram, J. L., Drouin, G., Barron, A. E., Slater, G. W., Electrophoresis 2005,26, 331-350.
5 Stellwagen, N. C., Gelfi, C., Righetti, P. G., Biopolymers 1997, 42, 687-703.
6 Stellwagen, N. C., Stellwagen, E., Electrophoresis 2002, 23, 1935-1941.
7 Olivera, B. M., Baine, P., Davidson, N., Biopolymers 1964, 2, 245-257.
8 Vökel, A. R., Noolandi, J., Macromolecules 1995, 28, 8182-8189.
9 Mayer, P., Slater, G. W., Drouin, G., Anal. Chem. 1994, 66,1777-1780.
10 Heller, C., Slater, G. W., Mayer, P., Dovichi, N., Pinto, D., Viovy, J.-L., Drouin, G., J. Chrom A 1998, 806,113-121.
11 Viovy, J.-L., Rev. Mod Phys. 2000, 72, 813-872.
12 Vreeland W. N., Desruisseaux, C., Karger, A. E., Drouin, G., Slater, G. W., Barron, A., Anal. Chem. 2001, 73, 1795-1803.
13 McCormick, L. C., Slater, G. W., Karger, A. E., Vreeland, W. N., Barron, A. E., Desruisseaux, C., and Drouin, G., J. Chrom. A 2001, 924, 43-52.
14 Long, D., Dobrynin, A. V., Rubinstein, M., Ajdari, A., J. Chem. Phys. 1998, 108, 1234-1244.
15 Sinton, D., Escobedo-Canseco, C., Ren, L., Li, D., Journal of Colloid and Interface Science 2002, 254, 184-189.
16 Ghosal, S., Electrophoresis 2004, 25, 214-228.
17 Potocek, B., Gas, B., Keandler, E., Stedry, M., J. Chrom. A 1995, 709, 51-62.
18 Gas, B., Stedry, M., Kenndler, E., J. Chrom. A 1995, 709, 63-68.
19 Kasicka, V., Prusik, Z., Sazelova, P., Chiari, M., Miksik, I., Deyl, Z., J. Chrom. B 2000, 741, 43-54.
20 Kasicka, V., Prusik, Z., Sazelova, P., Brynda, E., Stejskal, J., Electrophoresis 1999, 20, 2484-2492.
21 Hartley, N. K., Hayes, M. A., Anal. Chem. 2002, 74, 1249-1255.

1. A method for separation of polymer molecules in solution according to their relative size, each polymer molecule comprising an end-label at or near one or both ends thereof, the method comprising the steps of:
(1) subjecting the polymer molecules in solution to electrophoresis;
(2) subjecting the polymer molecules in solution during electrophoresis to an electroosmostic flow, such that the polymer molecules migrate in the solution at different rates, and optionally in different directions, according to their mobility in the solution.

2. The method of claim I, wherein in step (2) the speed of electroosmotic flow is about equal to a speed of unlabelled DNA subjected to the electrophoresis of step (1).

3. The method of claim 1, wherein in step (2) the speed of electroosmotic flow is less than a speed of unlabelled DNA subjected to the electrophoresis of step (1).

4. The method of claim 1, wherein at least some of the polymer molecules migrate in opposite directions according to a relative force upon them caused by said electrophoresis and said electroosmostic flow.

5. The method of claim 1, wherein said solution is retained in a capillary tube.

6. The method of claim 5, wherein the capillary tube comprises an internal wall that is uniformly charged, and wherein the solution at both ends of the capillary tube is at about the same pressure.

7. The method of claim I, wherein in step (2) the electroosmotic flow is constant and causes a countercurrent to a mobility of at least some of the polymer molecules during electrophoresis.

8. The method of claim 1, wherein the polymer molecules are separated with a polymer unit resolution Sₘ calculated according to equation (8):
$${S}_{m} ( {M}_{c} , {\tilde{μ}}_{EOF} ) ≡ \frac{{FWHM}_{t}}{\vert \partial t / \partial {M}_{c} \vert} \qquad ( 8 )$$
wherein the components of equation 8 are herein defined.

9. The method of claim 1, wherein the polymer molecules are polynucleotides.

10. The method of claim 9, wherein the polynucleotides are separated with a resolution of one nucleotide or less.

11. The method of claim 10, wherein the polynucleotides are derived from sequencing reactions for a DNA, the method further comprising a step of:
(3) deducing a nucleotide in said DNA corresponding to each polymer molecule, so as to deduce a sequence of the DNA.

12. An apparatus for separation of polymer molecules in solution according to their relative size, each polymer molecule comprising an end-label at one or both ends thereof, the apparatus comprising:
(1) electrophoresis means for subjecting the polymer molecules in the solution to electrophoresis;
(2) electroosmostic flow means for subjecting the polymer molecules in the solution to an electroosmostic flow during electrophoresis;
whereupon subjecting the polymer molecules to simultaneous electrophoresis and electroosmotic flow, the polymer molecules migrate in the solution at different rates, and optionally in different directions, according to their mobility in the solution.

13. A method for sequencing a section of a DNA molecule, the method comprising the steps of:
(a) synthesizing a first plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific adenine base in said section of DNA;
(b) synthesizing a second plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific cytosine base in said section of DNA;
(c) synthesizing a third plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific guanine base in said section of DNA;
(d) synthesizing a fourth plurality of ssDNA molecules each comprising a sequence identical to at least a portion at or near the 5′ end of said section of DNA, said ssDNA molecules having substantially identical 5′ ends but having variable lengths, the length of each ssDNA molecule corresponding to a specific thymine base in said section of DNA;
(e) attaching at least one chemical moiety to nucleotides at or near at least one end of said ssDNA molecules to generate end-labeled ssDNAs; and
(f) subjecting each plurality of end labeled ssDNA molecules to free-solution electrophoresis;
(g) subjecting the polymer molecules in solution during electrophoresis to an electroosmostic flow such that the polymer molecules migrate in the solution at different rates, and optionally in different directions, according to their mobility in the solution; and;
(h) identifying the nucleotide sequence of the section of DNA in accordance with the relative electrophoretic mobilities of the end labeled ssDNAs in each plurality of ssDNAs;
wherein any of steps (a), (b), (c), and (d) may be performed in any order or simultaneously;
whereby each end label imparts increased hydrodynamic friction to at least one end of each end-labeled ssDNA thereby to facilitate separation of the end-labeled ssDNAs according to their electrophoretic mobility.

14. The method of claim 14, wherein the ssDNAs are uncharged chemical moieties.

15. The method of claim 14, wherein the ssDNAs are selected from among polypeptides and polypeptoids.

16. The method of claim 14, wherein the ssDNAs are selected from the group consisting of Streptavidin, or a derivative thereof, N-methoxyethylglycine (NMEG)-based polymers comprising up to 300 preferably 100 monomer units, and a molecule consisting of a poly(NMEG) backbone optionally grafted with oligo(NMEG) branches

17. The method according to claim 14, wherein the section of DNA comprises less than 2000 nucleotides.

18. The method according to claim 17, wherein the section of DNA comprises less than 500 nucleotides.

19. The method according to claim 18, wherein the section of DNA comprises less than 100 nucleotides.

20. An apparatus for sequencing a DNA molecule by carrying out at least steps (f), (g), and (h) of the method of claim 13, thereby to separate ssDNAs produced in steps (a), (b), (c), and (d) according to their relative size, each ssDNA comprising an end-label at one or both ends thereof, the apparatus comprising:
(1) electrophoresis means for subjecting the ssDNAs to electrophoresis;
(2) electroosmostic flow means for subjecting the ssDNAs to an electroosmostic flow during said electrophoresis;
whereupon subjecting the ssDNAs to simultaneous electrophoresis and electroosmotic flow, the ssDNAs migrate in the solution at different rates, and optionally in different directions, according to their mobility in the solution; and
(3) nucleotide identification means for identifying each nucleotide in a sequence of said DNA molecule according to a mobility of the ssDNAs in the solution.
